(19) United States
(12) Patent Application Publication
(10) Pub. No.: US 2001/0002255 A1
(43) Pub. Date: May 31, 2001

(54) Medicaments

(75) Inventors: Graham R. Foster, London (GB); Howard C. Thomas, London (GB)

Correspondence Address: Nixon & Vanderhye P.C., 8th Floor, 1100 N. Glebe Rd., Arlington, VA, 22201, US

(73) Assignee: Imperial College of Science Technology & Medicine

(21) Appl. No.: 09/738,290

(22) Filed: Dec. 18, 2000

Related U.S. Application Data
(63) Continuation of application No. 09/426,724, filed on Oct. 26, 1999.
Application No. 09/426,724 is a continuation of application No. 09/109,077, filed on Jul. 2, 1998, now abandoned.
Application No. 09/109,077 is a continuation of application No. PCT/GB98/00269, filed on Jan. 29, 1998.

(30) Foreign Application Priority Data
Jan. 31, 1997 (GB) 9702021.8

(51) Int. Cl. A61K 38/21; A61K 38/00

(52) U.S. Cl. 424/85.7; 514/12

(57) ABSTRACT

The use of an IFN-α subtype is provided for the preparation of a medicament to enhance the T-cell immune response in therapy of cancer, bacterial or parasitic infection or systemic viral infection amongst other disease conditions. Also provided are pharmaceutical formulations which include such sub-types of IFN-α and methods of treatment, including treatment of cancer, bacterial or parasitic infection or systemic viral infection amongst other disease conditions.

The present invention relates to the use of Interferon-α (IFN-α) subtypes, particularly IFN-α₈, in the preparation of medicaments to treat certain diseases as well as methods of immunomodulation comprising administration of one or more IFN-α subtypes.

Type I interferons (IFN) are a family of closely related glycoproteins containing many IFN-α subtypes and one IFN-β subspecies. At least 13 different human IFN-α subtypes have been identified by analysis of human cDNA libraries and by protein analysis of the IFNs produced by stimulated lymphoblastoid cells. The reasons for this heterogeneity are not yet known. Previous studies have suggested that all Type I IFNs bind to an identical receptor and therefore have identical effects. However a mutant cell line that responds only to IFN-β and interferon-α₈ but not other IFN-α subtypes has been identified showing that these two IFN subspecies either bind to a different receptor or bind in a different way and may therefore have different effects. Molecular analysis of the human Type I IFN receptor thus suggests that the receptor may be able to distinguish between different IFN subtypes.

A number of studies have compared the effects of different IFN-α subtypes on the antiviral activities of human cell lines. Zoon et al (J. Biol. Chem. 267:15210-16 (1992) studied IFNs that were obtained from HPLC purification of natural IFN and found no gross differences in their antiviral activities. However, Sperber et al, J. Interferon. Res. 12 363-368 (1992) examined the effects of different recombinant IFN-α subtypes on cells infected with the human immunodeficiency virus (HIV) and found marked differences in their antiviral properties. WO95/24212 disclosed that different IFN-α subtypes were effective antiviral agents in different cell types.

Thus, it is possible to target viral infections in say the liver by the use of particular subtypes, eg IFN-α₈.

B cells or B lymphocytes are a subset of lymphocytes found in secondary lymphoid organs as well as circulating in the blood. They are characterised by the possession of antigen-specific cell surfaceimmunoglobulin molecules of a single antigen-binding specificity which act as receptors for antigen. The interaction of antigen with the cell-surface immunoglobulin is in part responsible for subsequent proliferation of the B cells and their development into antibody-secreting plasma cells. We have found that B cell proliferation can be induced by certain IFN-α subtypes.

T cells are a class of lymphocytes which mediate immune recognition and effect cell-mediated immune responses. In the course of a normal immune response, the binding of a ligand (normally the antigenic complex of peptide and MHC molecule) to the T cell receptor complex (TCR-CD3) on the surface of a T cell initiates intracellular changes, usually leading to proliferation of the T cell concerned and the production of lymphokines.

We have now found that IFN-α subtypes are capable of manipulating the T cell response and particularly enhancing the T cell immune response.

Thus, in a first aspect the present invention provides the use of an IFN-α subtype in the preparation of a medicament to enhance the T cell immune response. In particular, IFN-α₈ is used.

In view of the ability of IFN-α subtypes to enhance the T cell immune response it is possible to use them in the preparation of medicaments to treat certain disese states. Thus, in further aspects the present invention provides:

i) the use of an IFN-α subtype in the preparation of a medicament for the treatment of cancer;

ii) the use of an IFN-α subtype in the preparation of a medicament for the treatment of a bacterial or parasitic infection; and

iii) the use of an IFN-α subtype in the preparation of a medicament to treat a systemic viral infection.

Although WO95124212 disclosed the use of certain IFN-α subtypes in the treatment of viral infections in certain cell types, the present invention relates to their use to treat viral infections which are effectively “systemic”, ie viral infections which affect more than one cell or tissue type.

Methods for the treatment of the above-noted conditions are also included within the scope of the present invention. Such methods will comprise administration of an effective amount of an IFN-α subtype, in particular IFN-α₈, to the subject.

In yet further aspects the present invention provides:

i) a pharmaceutical formulation for use in enhancing the T cell immune response which comprises an IFN-α subtype, optionally together with one or more pharmaceutically acceptable carriers, excipients or diluents;

ii) a pharmaceutical formulation for use in the treatment of cancer which comprises an IFN-α subtype, optionally together with one or more pharmaceutically acceptable carriers, excipients or diluents;

iii) a pharmaceutical formulation for use in the treatment of bacterial or parasitic infections which comprises an IFN-α subtype, optionally together with one or more pharmaceutically acceptable carriers, excipients or diluents; and

iv) a pharmaceutical formulation for use in the treatment of systemic viral infections which comprises an IFN-α subtype, optionally together with one or more pharmaceutically acceptable carriers, excipients or diluents.

In all the above cases the preferred IFN-α subtype is IFN-α₈.

Pharmaceutical formulations may be presented in unit dose forms containing a predetermined amount of active ingredient per dose. The precise dose will of course depend on the condition being treated, the route of administration and the age, weight and condition of the patient.

Pharmaceutical compositions within the scope of the present invention may include one or more of the following; preserving agents, solubilising agents, stabilising agents, wetting agents, emulsifiers, sweeteners, colorants, odourants, salts, buffers, coating agents or antioxidants. They may also contain therapeutically active agents.

Pharmaceutical compositions within the scope of the present invention may be adapted for a administration by any appropriate route, for example by the oral (including buccal or sublingual), rectal, nasal, topical (including buccal, sublingual or transdermal), vaginal or parenteral (including subcutaneous, intramuscular, intravenous or intradermal) route. Such a composition may be prepared by any method known in the art of pharmacy, for example by admixing the active ingredient with a carrier under sterile conditions.

Various routes of administration will now be considered in greater detail:

(I) Oral Administration

Pharmaceutical compositions adapted for oral administration may be provided as capsules or tablets; as powders or granules; as solutions, syrups or suspensions (in aqueous or non-aqueous liquids); as edible foams or whips; or as emulsions.

Tablets or hard gelatine capsules may comprise lactose, maize starch or derivatives thereof, stearic acid or salts thereof.

Soft gelatine capsules may comprise vegetable oils, waxes, fats, semi-solid, or liquid polyols etc.

Solutions and syrups may comprise water, polyols and sugars. For the preparation of suspensions oils (e.g. vegetable oils) may be used to provide oil-in-water or water-in-oil suspensions.

(ii) Transdermal Administration

Pharmaceutical compositions adapted for transdermal administration may be provided as discrete patches intended to remain in intimate contact with the epidermis of the recipient for a prolonged period of time. For example, the active ingredient may be delivered from the patch by iontophoresis (Iontophoresis is described in Pharmaceutical Research, 3(6):318 (1986)).

(iii) Topical Administration

Pharmaceutical compositions adapted for topical administration may be provided as ointments, creams, suspensions, lotions, powders, solutions, pastes, gels, sprays, aerosols or oils.

For infections of the eye or other external tissues, for example mouth and skin, a topical ointment or cream is preferably used. When formulated in an ointment, the active ingredient may be employed with either a paraffinic or a water-miscible ointment base. Alternatively, the active ingredient may be formulated in a cream with an oil-in-water base or a water-in-oil base.

Pharmaceutical compositions adapted for topical administration to the eye include eye drops. Here the active ingredient can be dissolved or suspended in a suitable carrier, e.g. in an aqueous solvent.

Pharmaceutical compositions adapted for topical administration in the mouth include lozenges, pastilles and mouthwashes.

(iv) Rectal Administration

Pharmaceutical compositions adapted for rectal administration may be provided as suppositories or enemas.

(v) Nasal Administration

Pharmaceutical compositions adapted for nasal administration which use solid carriers include a coarse powder (e.g. having a particle size in the range of 20 to 500 microns). This can be administered in the manner in which snuff is taken, i.e. by rapid inhalation through the nose from a container of powder held close to the nose.

Compositions adopted for nasal administration which use liquid carriers include nasal sprays or nasal drops. These may comprise aqueous or oil solutions of the active ingredient.

Pharmaceutical compositions adapted for administration by inhalation include fine particle dusts or mists, which may be generated by means of various types of apparatus, e.g. pressurised aerosols, nebulizers or insufflators. Such apparatus can be constructed so as to provide predetermined dosages of the active ingredient.

(vi) Vaginal Administration

Pharmaceutical compositions adapted for vaginal administration may be provided as pessaries, tampons, creams, gels, pastes, foams or spray formulations.

(vii) Parenteral Administration

Pharmaceutical compositions adapted for parenteral administrations include aqueous and non-aqueous sterile injectable solutions or suspensions. These may contain antioxidants, buffers, bacteriostats and solutes which render the compositions substantially isotonic with the blood of an intended recipient. Other components which may be present in such compositions include water, alcohols, polyols, glycerine and vegetable oils, for example. Compositions adapted for parenteral administration may be presented in unit-dose or multi-dose containers, for example sealed ampoules and vials, and may be stored in a freeze-dried (lyophilised) condition requiring only the addition of a sterile liquid carrier, e.g. sterile water form injections, immediately prior to use. Extemporaneous injection solutions and suspensions may be prepared from sterile powders, granules and tablets.

Preferred unit dosage formulations are those containing a daily dose or sub-dose, as herein above recited, or an appropriate fraction thereof, of an active ingredient.

In a final aspect the present invention provides a method for enhancing the T cell immune response which comprises administering to a subject an effective amount of an IFN-α subtype.

The invention will now be described by reference to the following example which should not be construed as in any way limiting the scope of the invention.

The examples refer to the figures in which:

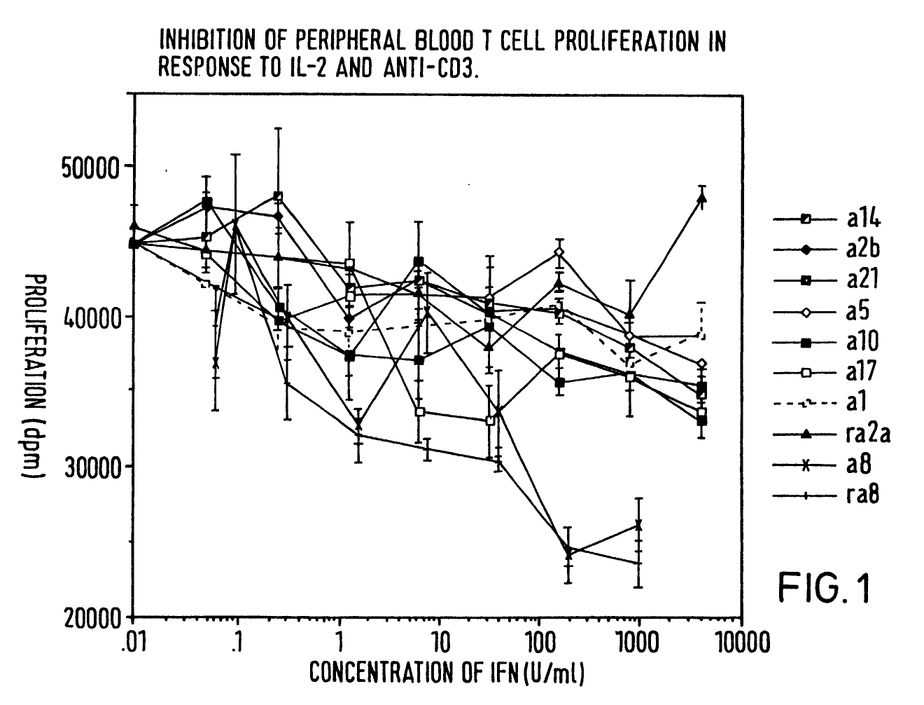

FIG. 1: shows inhibition of peripheral blood T cell proliferation in response to IL-2 and anti-CD3; and

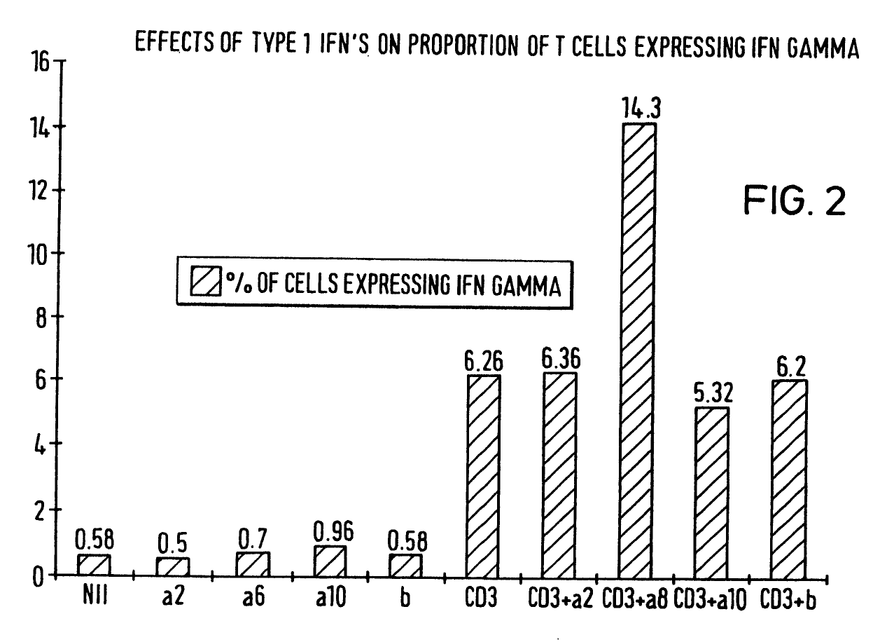

FIG. 2: shows the effect of various IFN-α subtypes on the production of IFNγ by T cells in the presence or absence of anti-CD3 antibodies plus IL-2.

### EXAMPLE 1

Peripheral blood T cells were purified by Ficoll density gradient centrifugation and E rosetting with sheep red blood cells (SRBC) to separate B and T cells. The T cells were recovered from the rosettes by lysis of the SRBC and were cultured at 1×10⁶ cells/ml in RPMI 1640 medium with 10% FCS and gentamycin for three days.

³H thymidine was added for the last 8 hours culture and incorporation was measured by scintilllation counting. The cells were stimulated with anti-CD3 (UCHT1) at 2 μg/ml. Under these conditions none of the IFN-α subtypes induced significant proliferation of the cells (data not shown).

However, when the T cells were stimulated with anti-CD3 and IL-2, inhibition of the IL-2 induced proliferation could be seen (see FIG. 1). IFN-α₈ was the most effective, with the other subtypes inhibiting to a lesser extent with the exception of IFN-α¹ which was inactive in the assay.

### EXAMPLE 2

Peripheral blood T cells were purified as in example 1. To investigate stimulation the cells were exposed to IFNα subtypes in the presence or absence of anti-CD3 antibodies (UCHT-1) plus IL-2 and IFNγ production was measured using standard intracellular staining techniques. The results are shown in FIG. 2 and indicate that anti-CD3 plus IL-2 antibodies plus IFN-α₈ caused an increase in the proportion of cells producing IFNγ.

1. The use of an IFN-α subtype in the preparation of a medicament to enhance the T cell immune response.

2. The use of an IFN-α subtype in the preparation of a medicament for the treatment of cancer.

3. The use of an IFN-α subtype in the preparation of a medicament for the treatment of a bacterial or parasitic infection.

4. The use of an IFN-α subtype in the preparation of a medicament to treat a systemic viral infection.

5. The use as claimed in any one of claims 1 to 4 wherein the subtype is IFN-α₈.

6. A pharmaceutical formulation for use in enhancing the T cell immune response which comprises an IFN-α subtype, optionally together with one or more pharmaceutically acceptable carriers, excipients or diluents.

7. A pharmaceutical formulation for use in the treatment of cancer which comprises an IFN-α subtype, optionally together with one or more pharmaceutically acceptable carriers, excipients or diluents.

8. A pharmaceutical formulation for use in the treatment of bacterial or parasitic infections which comprises an IFN-α subtype, optionally together with one or more pharmaceutically acceptable carriers, excipients or diluents.

9. A pharmaceutical formulation for use in the treatment of systemic viral infections which comprises an IFN-α subtype, optionally together with one or more pharmaceutically acceptable carriers, excipients or diluents.

10. A pharmaceutical formulation as claimed in any one of claims 6 to 9 wherein the subtype is IFN-α₈.

11. A method of enhancing a subject's T cell immune response which comprises administering an effective amount of an IFN-α subtype to the subject.

12. A method for the treatment of cancer which comprises administering an effective amount of an IFN-α subtype to the subject.

13. A method for the treatment of a bacterial or parasitic infection which comprises administering an effective amount of an IFN-α subtype to the subject.

14. A method for the treatment of a systemic viral infection which comprises administering an effective amount of an IFN-α subtype to the subject.

15. A method as claimed in any one of claims 11 to 14 wherein the subtype is IFN-α₈.
